(19) United States
(12) Patent Application Publication
(10) Pub. No.: US 2010/0305064 A1
(43) Pub. Date: Dec. 2, 2010

(54) TOPICAL SKIN CARE COMPOSITIONS

(76) Inventor: Star M. Walsh, Upper Black Eddy, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/788,908

(22) Filed: May 27, 2010

Related U.S. Application Data
(63) Continuation-in-part of application No. 12/731,193, filed on Mar. 25, 2010.
(60) Provisional application No. 61/182,284, filed on May 29, 2009.

(51) Int. Cl. A61K 8/92; A61Q 19/00

(52) U.S. Cl. 514/60

(57) ABSTRACT

The present invention relates to an oil-in-water emulsion. The emulsion comprises a starch and a quaternary ammonium salt. The concentration of the quaternary ammonium salt is greater than about 0.5 percent by weight of the composition, the concentration of starch is greater than about 3 percent by weight of the composition, and the combined concentration of the quaternary ammonium salt plus the starch is greater than about 4 percent by weight of the composition. Methods of treating the skin are provided.

This application claims the benefit of U.S. Provisional Application Ser. No. 61/182,284 filed on May 29, 2009; and is a continuation in-part of U.S. patent application Ser. No. 12/731,193 filed on Mar. 25, 2010, the complete disclosures of which are hereby incorporated herein by reference for all purposes.

### FIELD OF THE INVENTION

The present invention relates to topical skin care compositions in the form of oil-in-water emulsions having improved skin-feel properties.

### BACKGROUND OF THE INVENTION

The use of silicones in skin care products is well known. Silicones are known to provide a skin feel that can take the form of one or more of various pleasant sensations including, for example, a feeling of richness, smoothness, and/or a “cushiony” feel. However, silicones are expensive and not always easy to formulate. As such, the inventors have recognized that it is desirable to have skin care compositions that provide a “silicone-like,” cushiony skin feel, yet have zero or reduced levels of silicones.

Accordingly, the inventors have now discovered that oil-in-water emulsion compositions comprising a starch and a quaternary ammonium salt provide such cushiony skin feel without the need for silicones.

### SUMMARY OF THE INVENTION

The present invention is directed to a composition comprising an oil-in-water emulsion, comprising greater than about 0.5 percent by weight of a starch, and greater than about 3 percent by weight of a quaternary ammonium salt, wherein the combined concentration of the quaternary ammonium salt plus the starch is greater than about 4 percent by weight.

The quaternary ammonium salt is defined by the Structure I:

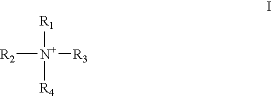

wherein at least one of R₁, R₂, R₃, and R₄ comprises a hydrophobic moiety.

### DETAILED DESCRIPTION OF THE INVENTION

The present invention meets the aforementioned need and overcomes the disadvantages of the prior art. In particular, it has been discovered that a quaternary ammonium salt can be combined with a starch in a composition to form an oil-in-water emulsion that has increased cushiony feel when applied to the skin. The oil in water emulsion is generally phase stable, i.e., the emulsion has a continuous aqueous phase and a discontinuous oil phase that do not separate into two or more distinct layers composed of hydrophilic components and lipophilic constituents when allowed to remain in a container at 25° C. for two weeks.

All percentages included herein are percentages by weight, based on total weight of the composition. Furthermore, as used herein, “substantially free” means the composition contains less than about 1, such as less than about 0.1, e.g., less than about 0.01 weight percent of an ingredient.

Any of a variety of suitable quaternary ammonium salts defined by Structure I may be used in the composition of the present invention. By “quaternary ammonium salt,” it is meant an amine having a quaternized nitrogen that has been substituted with at least one hydrophobic moiety.

As used herein, “quaternized nitrogen” means a charged polyatomic cation as represented in Structure I. In general, quaternary ammonium salts have a permanent, pH-independent charge. One skilled in the art will recognize that this pH-independent charge behavior is in contrast to primary ammonium cations (RNH₃⁺), secondary ammonium cations (R₂NH₂⁺) and tertiary ammonium cations (R₃NH⁺).

In quaternary ammonium salts useful in the present invention, at least one, and desirably at least two, of R₁, R₂, R₃, and R₄ are or include a hydrophobic moiety. In other words, in certain embodiments, the quaternary ammonium salt may include two distinct hydrophobic moieties, each of the two distinct hydrophobic moieties being separately pending from a quaternized nitrogen.

The remainder of the R₁, R₂, R₃ and R₄ groups are, in certain embodiments, also “substituted,” i.e. they do not each consist only of a hydrogen atom, but the substitution does not necessarily include a hydrophobic moiety. As such, the remainder of the R₁, R₂, R₃ and R₄ groups may be selected from the group consisting of, for example, methyl, ethyl, propyl and hydroxyl groups. In one embodiment, the remainder of the R₁, R₂, R₃ and R₄ groups are methyl groups.

In a particular embodiment, two of the R₁, R₂, R₃ and R₄ groups include hydrophobic moieties. For example, R₂ and R₃ may each be hydrophobic moieties. In one embodiment, the hydrophobic moieties in R₂ and R₃ are identical. In yet another embodiment, R₂ and R₃ have different carbon chain lengths. For example, R₂ may have a carbon chain length different from R₃ and that is at least 4, such as at least 6, such as at least 8 carbon atoms. In another embodiment, three or four of the R₁, R₂, R₃ and R₄ groups include hydrophobic moieties.

The term “hydrophobic moiety” as used in this specification to describe the above R groups on the quarternary ammonium salt, means any nonpolar, generally water-insoluble group containing four or more carbon atoms. Certain hydrophobic moieties include moieties comprising eight or more carbon atoms, such as 10 or more carbon atoms, e.g., at least 12 carbon atoms, such as 14 or more carbon atoms. The carbon atoms may be arranged in an uninterrupted fashion, e.g., each of the carbon atoms other than those terminating the particular group of carbon atoms has neighboring carbon atoms. Nonlimiting examples of hydrophobic groups include any alkyl, aryl or arylalkyl group, e.g., saturated or unsaturated linear, branched, cyclic, or aromatic hydrocarbon species. Functionalities that may be included in the hydrophobic group may be selected from the group consisting of, for example, ether, ester, ketone, urethane, carbamate and xanthate functionalities.

In one embodiment, the hydrophobic moiety includes an alkyl group having seven or more carbon atoms, such as 10 or more carbon atoms, e.g., 12 or more carbon atoms, such as 14 or more carbon atoms. To provide sufficient hydrophilic character, in certain embodiments of the invention, the quaternary ammonium salt has a hydrophile-liphophile balance that is about 9 or greater, such as about 10 or greater.

In one embodiment, the quaternary ammonium salt is a dialkyl ammonium compound, such as a dialkyl ammonium chloride, such as distearyl dimethylammonium chloride, sold under the trade name VARISOFT TA-100, commercially available from EVONIK Goldschmidt/Degussa GmbH of Essen, Germany. Other suitable quaternary ammonium compounds include “diester quats” that have two hydrophobic moieties, each of which include an alkyl group (tallow, palm oil, or rapeseed oil-derived) and an ester functional group. Another suitable quaternary ammonium salt is distearoylethyl dimonium chloride, sold under the trade name VARISOFT EQ65, also commercially available from EVONIK GmbH. Other examples are sold under the trade name STEPANTEX, e.g., STEPANTEX VK-90. Another suitable quaternary ammonium compound is a “diamidoamine quaternary” sold under the trade name ACCOSOFT. Another suitable quaternary ammonium compound is a “dialkyldimethyl ammonium chloride” sold under the trade name BTC, e.g., BTC 2125M. Yet another suitable quaternary ammonium compound is an arylalkyldiemthyl ammonium chloride, such as an alkylbenzyldimethylammonium chloride, sold under the trade name STEPANQUAT. STEPANTEX, ACCOSOFT, BTC and STEPANQUAT products are available from Stepan Company of Northfield, Ill. The total concentration of quaternary ammonium salt in the composition is at least about 3% by weight, and may be from about 3% to about 10% by weight of the composition, e.g., from about 3% to about 8% by weight, such as from about 3% to about 7% by weight of the composition. One or more than one quaternary ammonium salt may be used.

One or more starches are also used in the composition. The starch may be of any variety of plant-derived polysaccharides having glucose repeat units joined by alpha acetal linkages to form amylose and/or amylopectin chains. Examples of suitable starches include those derived from such plants as corn, wheat, rice, tapioca, potato, sago, and the like. Such starches can be of a native variety or those developed by plant breeding or by gene manipulation. In an embodiment of the invention, the starches may include either the low amylase variety including less than about 5% amylose, high amylose starches including more than about 40% amylose, and/or combinations thereof. The starch may be of the granular or, so-called, “cooked” (dispersed) variety. Furthermore, the starch may be chemically modified such as by chemical crosslinking via e.g., ionic crosslinking (calcium, aluminum, or phosphate). Also, the starch may be chemically modified to include hydrophobic moieties, or other non-ionic moieties, e.g., hydroxypropyl. In a notable embodiment, the starch is crosslinked and substituted with one or more non-ionic moieties, such as hydrophobic moieties and/or hydroxypropyl moieties. An example of such crosslinked and substituted starches are hydroxypropyl starch phosphates. In another notable embodiment, the starch is absent of any substitutions of non-ionic moieties.

The starch may be of varying particle size. In certain embodiments, the starch has an average particle size that is from about 1 micron to about 100 microns, such as from about 5 microns to about 50 microns, e.g., from about 10 microns to about 40 microns.

An example of a suitable hydrophobically-modified starch is an aluminum starch octenyl succinate, available under the trade name DRY-FLO PURE (aluminum crosslinked, granular). Another suitable example of a non-ionic starch that is substituted with hydroxypropyl groups is a hydroxypropyl starch phosphate, available under the trade name STRUCTURE XL (phosphate crosslinked, dispersed, 100% amylopectin). Examples of such starches are available under the trade names NAVIANCE TAPIOCA, NAVIANCE INSTANT MAIZE and ZEA MAYS corn starch. DRY-FLO PURE and STRUCTURE XL are NAVIANCE starches commercially available from Akzo Nobel SPG LLC of Bridgewater, N.J.

The composition includes at least about 0.5% by weight starch, and may include from about 0.5% to about 5% starch, such as about 0.5% to about 5% starch, e.g., about 1.5% to about 5% by weight starch.

The inventors have found that high levels of hydrophobic agents may detract from the ability of the inventive compositions to provide cushiony skin feel. As such, in one notable embodiment, the composition includes relatively low levels of hydrophobic agents, e.g. less than about 20% by weight, such as less than about 15%, for example, less than about 10%.

By hydrophobic agents it is meant a molecule that meets one or more of the following three criteria: (a) has a carbon chain of at least six carbons in which none of the six carbons is a carbonyl carbon or has a hydrophilic moiety bonded directly to it; (b) has two or more alkyl siloxy groups; or (c) has two or more oxypropylene groups in sequence. A hydrophobic moiety may include linear, cyclic, aromatic, saturated or unsaturated groups.

One skilled in the art will recognize that “hydrophobic agents” does not include amphiphilic molecules such as emulsifiers, surfactants and other surface active compounds that have a solubility in pure deionized water of more than about 1%. By “water solubility” it is meant the maximum weight percentage of ingedient (relative to ingredient plus water) that can be placed into 100 grams deionized water and agitated so that a clear solution is obtained and remains visually homogeneous and substantially transparent at room temperature for 24 hours. Amphilphilic molecules that will be understood to be excluded from hydrophobic agents include those compounds that that have a solubility in pure deionized water of more than about 1% and include both of (a) a hydrophobic moiety defined above and (b) a hydrophilic moiety, such as anionic, cationic, zwitterionic, or nonionic group, that is polar, including sulfate, sulfonate, carboxylate, phosphate, phosphonates; ammonium, including mono-, di-, and trialkylammonium species, pyridinium, imidazolinium, amidinium, poly(ethyleneiminium); ammonioalkylsulfonate, ammonioalkylcarboxylate, amphoacetate; hydroxyl, and poly(ethyleneoxy)sulfonyl). Emulsifiers, surfactants and other surface active compounds are commonly used for emulsification and wetting rather than for film-formation, spreading and the like.

Hydrophobic agents used in the present invention are generally insoluble in water and are spreadable across the skin. Examples of suitable hydrophobic agents include emollients, such as mineral oils/waxes, including petrolatum, vegetable oils (glyceryl esters of fatty acids, triglycerides), waxes and other mixtures of fatty esters. Examples of such emollients include, without limitation, isopropyl palmitate, cetyl alcohol, isocetyl alcohol, caprylic/capric triglycerides, methyl undecylate, and dicaprylyl ether, and di-PPG 3 myristyl ether adipate. Another class of suitable hydrophobic agents for use in the present invention is organic ultraviolet (UV) filters.

Organic UV filters that are useful hydrophobic agents in the present invention are compounds that absorb radiation in the UV range. Examples of organic UV filters include, without limitation, 3-benzylidene camphor, specifically 3-benzylidene norcamphor and derivatives thereof, e.g. 3-(4-methylbenzylidene) camphor; 4-aminobenzoic acid derivatives, specifically 4-(dimethylamino)benzoic acid-2-ethylhexyl esters, 4-(dimethylamino)benzoic acid-2-octyl esters and 4-(dimethylamino)benzoic acid amylesters; esters of cinnamonic acid, in particular 4-methoxycinnamonic acid-2-ethylhexylester, 4-methoxycinnamonicacid propylester, 4-methoxycinnamonic acid isoamyl ester, 2-cyano-3,3-phenylcinnamonic acid-2-ethylhexyl ester (octocrylene); esters of salicylic acid, i.e., salicylic acid-2-ethylhexylester, salicylic acid-4-isopropylben-zyl ester, salicylic acid homomethyl ester; derivatives of benzophenones, in particular 2-hydroxy-4-methoxybenzophenone, 2-hydroxy-4-methoxy-4′-methylbenzophenone, 2,2′-dihydroxy-4-methoxybenzophenone; esters of benzalmalonic acid, in particular 4-methoxybenzmalonic acid di-2-ethylhexyl ester; triazine derivatives, for example 2, 4,6-trianilino-(p-carbo-2′-ethyl-1′-hexyloxy)-1,3,5-triazine and octyltriazone; or benzoic acid, 4,4′-[[6-[[[(1,1-dimethylethyl)amino]carbonyl]phenyl]amino]-1,3,5-triazine-2,4-diyl]diimino]bis-, bis (2-ethylhexyl) ester (UVASORB HEB); propane-1,3-diones, for example, 1-(4-tert.butylphenyl)-3-(4′-methoxyphenyl) propane-1,3-dione; ketotricyclo (5.2.1.0) decane derivatives; derivatives of benzoylmethane, for example, 1-(4′-tert.butylphenyl)-3-(4′-methoxyphenyl)propane-1,3-dione, 4-tert-butyl-4′-methoxydibenzoylmethane (PARSOL 1789), 1-phenyl-3-(4′-isopropylphenyl)-propane-1,3-dione, derivatives of benzoic acid 2-(4-diethylamino-2-hydroxybenzoyl)-benzoic acid hexylester (UVINUL A+), or 1H-benzimidazole-4,6-disulfonic acid, 2,2′-(1,4-phenylene)bis-, disodium salt (NEO HELOPAN AP); and benzotriazoles, in particular the benzotriazole derivative known as 2,2′-methylene-bis-(6-(2H-benzotriazole-2-yl)-4-(1,1,3,3-tetramethylbutyl)-phenol) [INCI: Bisoctyltriazol], which is commercially available under the tradename TINOSORB M from CIBA Chemicals. Another useful benzotriazole derivative is 2-(2H-benzotriazole-2-yl)-4-methyl-6-[2-methyl-3-[1,3,3,3-tetramethyl-1-[(trimethylsilyl)oxy]disiloxanyl]propyl]-phenol (CAS-No.: 155633-54-8), also identified by the INCI name drometrizole trisiloxane and available from Chimex under the tradename MEXORYL XL.

Oil-soluble broadband filters include the asymmetrically substituted triazine derivatives. Of particular interest is 2,4-bis-{[4-(2-ethyl-hexyloxy)-2-hydroxyl-phenyl]}-6-(4-methoxyphenyl)-1,3,5-triazine (INCI: anisotriazine) that is commercially available under the tradename TINOSORB S from CIBA Chemicals.

Compositions of the present invention are oil-in water (O/W) emulsions. By O/W emulsions it is meant that a discontinuous oil phase is substantially homogenously suspended in a continuous aqueous phase. The continuous aqueous phase generally includes water (i.e., is ionically conductive), as well as other ingredients that are soluble, miscible or dispersed with the water. The concentration of water in the composition may be from about 10 weight percent to about 78 weight percent, such as from about 35 weight percent to about 65 weight percent, e.g., from about 40 weight percent to about 60 weight percent. The oil phase includes the hydrophobic agents, as well as other optional ingredients that are soluble, miscible or partitioned in with the hydrophobic agents.

The inventors have found that, in order to provide the desirable property of cushiony feel in compositions according to the present invention, the starch and the quaternary ammonium salt should be present in a total combined concentration of starch plus quaternary ammonium salt that is at least about 4%, e.g., at least about 5.5%, such as at least about 6.5%; such as from about 6.5% to about 9% by weight of the composition.

In another embodiment, the composition comprises an additional emulsifier that does not meet the requirements defined above to be a quaternary ammonium salt. The additional emulsifier is, in certain embodiments, a non-ionic emulsifier, present in a concentration of about 0.1 to about 5.0 weight percent, such as from about 1 to about 3 weight percent. Examples of suitable additional non-ionic emulsifiers include fatty alcohols such as cetearyl alcohol, fatty esters, Poloxamer 184, laureth-4, sorbitan esters such as sorbitan trioleate and sorbitan stearate, polyoxyethylene-(2) oleyl ether, polyoxyethylene ethers of fatty alcohols such as ceteareth-20, isocetheth-20, cetearyl glucoside, glyceryl oleate, trideceth-9, polyethylene glycol-40 hydrogenated castor oil; steareth-21, methylheptyl isostearate, and mixtures thereof.

In another embodiment, the composition has reduced levels of silicones. Silicones, as defined herein, are generally hydrophobic compounds having a silicon atom that is bonded to at least one oxygen atom and at least one carbon atom, e.g., a carbon atom that is a part of a methyl or other alkyl group. The silicones that the composition has reduced levels of are, in certain embodiments, liquids at room temperature, but in other embodiments may be solid at room temperature, such as silicone polymers and silicone gums. By “reduced levels” it is meant that the concentration of total silicones is less than about 3%, such as less than about 2% by weight of the composition. In certain embodiments, the composition is entirely free of silicones.

In one embodiment, the composition includes a humectant that serves to enhance spreadibility and/or moisture retention. Any of a variety of commercially available humectants, which are capable of providing moisturization and conditioning properties to the composition, are suitable for use in the present invention. Examples of suitable humectants nonexclusively include: 1) water soluble liquid polyols selected from the group comprising glycerine, propylene glycol (e.g., 1-2 or 1-3 propanediol), hexylene glycol, butylene glycol, dipropylene glycol, polyglycerols, and mixtures thereof; 2) polyalkylene glycols of the formula: HO—(R″O)_{b}—H, wherein R″ is an alkylene group having from about 2 to about 3 carbon atoms and b is an integer of from about 2 to about 10; 3) polyethylene glycol ether of methyl glucose of formula CH₃—C₆H₁₀O₅—(OCH₂CH₂)_{c}—OH, wherein c is an integer from about 5 to about 25; 4) urea; f) grain flours such as oat flour that has been milled to a particle size of about 100 microns and is suitable for forming colloidal dispersions in the inventive composition, and 5) mixtures thereof. In one embodiment, the humectant is a polyhydric alcohol such as glycerol or a propanediol such as propylene glycol. The humectant may be present in an amount of from about 1% to about 70% by weight in composition, such as from about 1% to about 40% by weight, e.g., from about 5% to about 30%, such as from about 1% to about 25%, based on the overall weight of the composition.

Compositions of the present invention may include a film-forming polymer in order to aid in film formation on the skin. Examples of film-forming agents include, but are not limited to or proteins and synthetic polymers such as polyesters, polyacrylics, polyurethanes, vinyl polymers, polysulfonates, polyureas, polyoxazolines, and the like. Specific examples of film-forming polymers include, for example, hydrogenated dimer dilinoleyl/dimethylcarbonate copolymer, available from Cognis Corporation of Ambler, Pa. under the trade name COSMEDIA DC; water-dispersible polyesters, including sulfopolyesters such those commercially available from Eastman Chemical as EASTMAN AQ 38S. The amount of film-forming polymer present in the composition may be from about 0.1% to about 5%, or from about 0.1% to about 3%, or from about 0.1% to about 2% by weight of the composition.

Compositions of the present invention may include a skin benefit agent. A benefit agent is any element, ion, or compound (e.g., a synthetic compound or a compound isolated from a natural source) or other chemical moiety in solid (e.g. particulate), liquid, or gaseous state that has a cosmetic or therapeutic effect on the skin, hair, mucosa, or teeth. As used herein, the term “benefit agent” includes any active ingredient such as a cosmetic or pharmaceutical, that is to be delivered into and/or onto the skin, hair, mucosa, or teeth at a desired location.

The benefit agents useful herein may be categorized by their therapeutic benefit or their postulated mode of action. However, it is to be understood that the benefit agents useful herein may, in some circumstances, provide more than one therapeutic benefit or operate via greater than one mode of action. Therefore, the particular classifications provided herein are made for the sake of convenience and are not intended to limit the benefit agents to the particular application(s) listed.

Examples of suitable benefit agents include those that provide benefits such as, but not limited to UV-filters, depigmentation agents; amino acids and their derivatives; anti-acne agents; anti-aging agents; anti-wrinkling agents; shine-control agents; antipruritic agents; hair growth inhibitor agents; anti-infective agents; anti-inflammatory agents; wound healing promoters; peptides, polypeptides and proteins; medicament agents; skin firming agents; vitamins; skin lightening agents; skin darkening agents; depilating agents; counterirritants; insecticides; poison ivy products; anti-diaper rash agents; prickly heat agents; herbal extracts; vitamin A and its derivatives; flavonoids; sensates and stress-reducing agents; anti-oxidants; keratolytics; inorganic pigments; and thickening agents. The amount of the benefit agent that may be used may vary depending upon, for example, the ability of the benefit agent to penetrate through the skin, nail, mucosa, or teeth; the specific benefit agent chosen, the particular benefit desired, the sensitivity of the user to the benefit agent, the health condition, age, and skin and/or nail condition of the user, and the like. In sum, the benefit agent is used in a “safe and effective amount,” which is an amount that is high enough to deliver a desired skin or nail benefit or to modify a certain condition to be treated, but is low enough to avoid serious side effects, at a reasonable risk to benefit ratio within the scope of sound medical judgment.

The pH of the present compositions is not critical, but to maximize suitability for the skin, the pH may be in a range from about 4.5 to about 8, for example, from about 4.5 to about 7, e.g., from about 4 to about 6.

Furthermore, in order to enhance stability of the composition, the concentration of electrolyte may be maintained at a total concentration of electrolyte (other than the quaternary ammonium salt) that is less than about 3%, e.g. less than about 2%, such as less than about 1% by weight of the composition. In certain other embodiments, the total concentration of electrolyte, including the quaternary ammonium salt is less than about 7%, such as from about 3.5% to about 7% by weight of the composition.

The composition is topically applied by means of directly laying on or spreading on outer skin, e.g., by use of the hands or an applicator such as a wipe, roller, or spray.

Compositions of the present invention are typically extrudable or dispensable from a package, such as may be caused to flow from the package to be applied directly or indirectly, topically to the body or another surface. Depending upon the particular function, compositions of present invention are desirably rubbed onto the skin and allowed to remain without rinsing.

Particularly suitable uses for compositions of the present invention include application onto human skin, such as for applications in which the composition is allowed to remain on the skin for at least about an hour without rising. Such “leave-on” applications for the inventive compositions include skin lotions such as skin moisturizers and skin conditioners, among other personal care applications. In certain embodiments of the invention, the inventive compositions are applied to mammalian (e.g., human) skin, such as facial skin or skin on various parts of the body (e.g., arms, legs, torso, etc.).

As discussed above, applicants have discovered unexpectedly that compositions of the present invention provide personal care products having high loading of hydrophobic agents without compromising aesthetics, such as is typified by high levels of skin whitening for comparable prior art compositions. Furthermore, compositions of the present inventions are phase-stable.

The following non-limiting examples further illustrate the claimed invention.

### EXAMPLES

### Example I

### Preparation of Inventive Examples and Comparative Examples

The following inventive examples and comparative examples were prepared, as shown in Tables 1-2. Amounts of ingredients are in percent by weight.

TABLE 1
Inventive Examples, Ex. 1-5

| Trade Name | INCI Name | Ex. 1 | Ex. 2 | Ex. 3 | Ex. 4 | Ex. 5 |
|---|---|---|---|---|---|---|
| Deionized | Water | 73.90 | 74.10 | 72.90 | 76.40 | 76.90 |
| Water | | | | | | |
| Glycerin | Glycerin | 8.00 | 8.00 | 8.00 | 8.00 | 8.00 |
| Cetyl Alcohol | Cetyl Alcohol | 2.50 | 2.50 | 2.50 | 2.50 | 2.50 |
| Cromollient | Di-PPG 3 Myristyl | 2.00 | 2.00 | 2.00 | 2.00 | 2.00 |
| DP3-A | Ether Adipate | | | | | |
| Miglyol 812 | Caprylic/Capric | 2.00 | 2.00 | 2.00 | 2.00 | 2.00 |
| | Triglycerides | | | | | |
| Promulgen D | Ceteareth-20 (and) | 0.00 | 0.00 | 2.00 | 0.00 | 2.00 |
| | Cetearyl Alcohol | | | | | |
| Kessco IPP | Isopropyl Palmitate | 2.00 | 2.00 | 2.00 | 2.00 | 2.00 |
| Varisoft | Distearyldimonium | 6.00 | 0.00 | 3.00 | 5.00 | 3.00 |
| TA-100 | Chloride | | | | | |
| Varisoft EQ-65 | Distearoylethyl- | 0.00 | 7.30 | 0.00 | 0.00 | 0.00 |
| | dimonium Chloride | | | | | |
| Benzyl alcohol | benzyl alcohol | 0.60 | 0.60 | 0.60 | 0.60 | 0.60 |
| Structure XL | Hydroxypropyl | 3.00 | 1.50 | 5.00 | 1.50 | 1.00 |
| | Starch Phosphate | | | | | |

TABLE 2
Inventive Examples, Ex. 6-7 and Comparative Examples, Comp. 1-4

| Trade Name | INCI Name | Ex. 6 | Ex. 7 | Comp. 1 | Comp. 2 | Comp. 3 | Comp. 4 |
|---|---|---|---|---|---|---|---|
| Deionized Water | Water | 75.15 | 77.4 | 79.40 | 76.65 | 78.15 | 77.90 |
| Glycerin | Glycerin | 8.00 | 8.00 | 8.00 | 8.00 | 8.00 | 8.00 |
| Cetyl Alcohol | Cetyl Alcohol | 2.50 | 2.50 | 2.50 | 2.50 | 2.50 | 2.50 |
| Dow Corning Q7- | Dimethicone | 1.25 | 0.00 | 0.00 | 1.25 | 1.25 | 0.00 |
| 9120 (20 CS) | | | | | | | |
| Cromollient DP3-A | Di-PPG 3 Myristyl | 2.00 | 2.00 | 2.00 | 2.00 | 2.00 | 2.00 |
| | Ether Adipate | | | | | | |
| Miglyol 812 | Caprylic/Capric | 2.00 | 2.00 | 2.00 | 2.00 | 2.00 | 2.00 |
| | Triglycerides | | | | | | |
| Kessco IPP | Isopropyl | 2.00 | 2.00 | 2.00 | 2.00 | 2.00 | 2.00 |
| | Palmitate | | | | | | |
| Promulgen D | Ceteareth-20 (and) | 0.00 | 0.00 | 2.00 | 0.00 | 2.00 | 0.00 |
| | Cetearyl Alcohol | | | | | | |
| Varisoft TA-100 | Distearyl dimonium | 5.00 | 5.00 | 0.00 | 5.00 | 0.00 | 5.00 |
| | chloride | | | | | | |
| Benzyl alcohol | Benzyl alcohol | 0.60 | 0.60 | 0.60 | 0.60 | 0.60 | 0.60 |
| Structure XL | Hydroxypropyl | 1.50 | 0.5 | 1.50 | 0.00 | 1.50 | 0.00 |
| | Starch Phosphate | | | | | | |

STRUCTURE XL, is available from Akzo Nobel (Bridgewater, N.J.), CROMOLLIENT DP3-A is available from Croda (Edison, N.J.), MIGLYOL 812 is available from Sasol Germany GmbH (Witten, Germany), KESSCO IPP is available from Brenntag North America, Inc (Reading, Pa.), and PROMULGEN D is available from Lubrizol Advanced Materials (Cleveland, Ohio).

The compositions were made by adding water and glycerin to a vessel, mixing and heating to 85C, adding (in particular examples) VARISOFT, cetyl alcohol, MIGLYOL, isopropyl palmitate, and (in particular examples) PROMULGEN and dimethicone; mixing at 85C for 15 minutes, removing from heat, allowing to cool while continuing to mix and allowing to cool to 30-35C. The formulas were then QS balanced with required amount of water, and (in particular examples) STRUCTURE XL was added and allowed to mix until uniform.

An additional Inventive Example, Ex. 8 was prepared, as shown in Table 3.

TABLE 3
Inventive Example, Ex. 8

| | | Weight |
|---|---|---|
| Trade Name | INCI Name | Percent |
| Deionized Water | Water | 66.50 |
| Glycerin | Glycerin | 8.00 |
| Natrosol Plus CS330 | Cetyl Hydroxyethylcellulose | 0.05 |
| Vivimed Cosvat | Chlorphenesin | 0.25 |
| Isofol 28 | Dodecylhexadecanol | 1.25 |
| Cetyl Alcohol | Cetyl Alcohol | 1.25 |
| Dow Corning | Dimethicone | 2.50 |
| Q7-9120 (20 CS) | | |
| Kessco IPP | Isopropyl Palmitate | 2.00 |
| Ceraphyl ICA | Isocetyl Alcohol | 2.00 |
| Beantree | Methylheptyl Isostearate | 2.00 |
| Varisoft TA-100 | Distearyldimonium Chloride | 5.00 |
| Brij 721 | Steareth-21 | 1.50 |
| Pulpactyl | Water (and) Butylene Glycol | 2.00 |
| | (and) Artemisia Abrotanum | |
| | Extract | |
| Neutrol TE | Tetrahydroxypropyl | 1.00 |
| | Ethylenediamine | |
| Deionized water | Water | 1.00 |
| Citric acid 50% Solution | Citric Acid | 0.50 |
| NAB Mushroom PF | Ganoderma Lucidum (Mushroom) | 0.30 |
| | Stem Extract (and) Lentinus | |
| | Edodes Extract (and) Algae | |
| | Extract (and) Phenoxyethanol | |
| Benzyl Alcohol | Benzyl Alcohol | 0.60 |
| Waterfall Mod 307431 | Fragrance | 0.30 |
| Structure XL | Hydroxypropyl Starch Phosphate | 2.00 |

The composition above was made by adding water, glycerin, cetyl hydroxyethylcellulose and chlorphenesin to a vessel, mixing and heating to 85C. In a separate container VARISOFT, cetyl alcohol, dodecylhexadecanol, methylhelptyl isostearate, steareth-21, isopropyl palmitate, and dimethicone were added, mixed and heated to 85C until all ingredients were uniform and melted. This phase was added to the water phase and mixing was continued at 85C for 10 minutes, after which the resulting batch was removed from the heat. The batch was allowed to cool to 30-40C while continuing to mix. Pulpactyl, neutrol TE, water, citric acid, NAB mushroom PF, benzyl alcohol and fragrance were added with continued mixing. The formula was then QS balanced with required amount of water, and STRUCTURE XL was added and allowed to mix until uniform.

An additional Comparative Example, Comp. 5 was prepared. Comparative Example, Comp. 5 was an oil-in-water emulsion that included no starch and no quaternary ammonium salt, but included about 22 percent hydrophobic agents, about 14.25% of which were silicones. Comparative Example also included about 2.5% electrolyte.

The compositions were each evaluated for cushiony skin feel in the following manner. Sixty microliters of product was placed in a 2×2 section of the volar forearm of each of four trained sensory expert panelists, and the panelist spread the test compositions in a circular motion in the 2×2 area at a rate of one rotation per second. Each panelist evaluated the compositions and rated them for amount of cushion. Cushion was rated during the third to fifth rotation on skin. Cushion is defined as the perceived distance between the skin and the product being rubbed on the skin. Cushion was rated on a 0-10 scale with 10 being the most distance, i.e., most cushion.

The magnitude of cushiony feel, i.e. the scores as assigned by panelits, is reported in Table 4. Samples are listed in order from highest score to lowest score. Cushion scores up to and including 3 are considered acceptable, with scores above 4 being particularly preferred. As described above, the base formulas were identical (other than starch and quaternary ammonium salt), except for Ex. 8 and Comp. 5 (the latter was a high silicone formula). Ex. 2 included a different quaternary ammonium salt than the other samples.

TABLE 4
Cushiony Feel Scores

| | | | Quat (%), |
|---|---|---|---|
| Example | Scores | Average Score | Starch (%) |
| Ex. 1 | 4.5, 6.25, 6.5, 6 | 5.8 | 6%, |
| | | | 3% |
| Ex. 2 | 5, 6, 6, 6 | 5.8 | 5%, |
| | | | 1.5% |
| Ex. 3 | 4, 4, 7.5, 5 | 5.1 | 3%, |
| | | | 5%, |
| Comp. 5 | 6, 6, 3.5. 2.5 | 5.1 | — |
| Ex. 4 | 5, 3.5, 6, 5 | 4.9 | 5%, |
| | | | 1.5% |
| Ex. 8 | 5, 3.5, 6, 5 | 4.9 | 5%, |
| | | | 2% |
| Ex. 5 | 4.5, 4,, 3, 4.25 | 3.9 | 3% |
| | | | 1% |
| Ex. 6 | 2, 3, 5, 4 | 3.5 | 5%, |
| | | | 1.5% |
| Ex. 7 | 3.5, 2.5, 2, 4 | 3.0 | 5% |
| | | | 0.5% |
| Comp. 1 | 2, 2, 3, 3 | 2.5 | 0% |
| | | | 1.5% |
| Comp. 2 | 2, 2.75, 3, 2 | 2.4 | 5% |
| | | | 0% |
| Comp. 3 | 1, 2, 3, 3 | 2.3 | 0% |
| | | | 1.5% |
| Comp. 4 | 1, 1.75, 2, 4 | 2.2 | 5% |
| | | | 0% |

As is evidenced by the results of the skin feel evaluation, the presence of both the quaternary ammonium salt and the starch are important for providing cushiony skin feel. When either of these ingredients is absent, cushiony feel falls to low levels. Furthermore, the cushiony feel is particularly enhanced when, for example, the concentration of starch is at least about 1.5%, and/or when the combined concentration of starch and quaternary ammonium salt is at least about 6.5% percent.

It is particularly surprising that the degree of cushiony feel that can be achieved is, in certain embodiments, as high or higher than Comparative Example, Comp. 5, which has over 14% silicone. For example, Inventive Examples Ex. 1-3 provide this enhanced feel with no silicone at all.

What is claimed is:

1. A composition comprising an oil-in-water emulsion, comprising:
greater than about 0.5 percent by weight of a starch; and
greater than about 3 percent by weight of a quaternary ammonium salt defined by the Structure I: 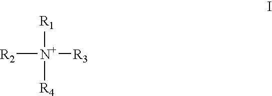
wherein at least one of R₁, R₂, R₃, and R₄ comprises a hydrophobic moiety; and wherein the combined concentration of the quaternary ammonium salt plus the starch is at least about 4 percent by weight, based on total weight of the composition.

2. The composition of claim 1, wherein the starch is present in a concentration of about 0.5 percent to about 5 percent by weight of the composition.

3. The composition of claim 1, wherein the starch is present in a concentration of about 1.5 percent to about 5 percent by weight of the composition.

4. The composition of claim 1, wherein the quaternary ammonium salt is present in a concentration of about 3 percent to about 10 percent by weight of the composition.

5. The composition of claim 1, wherein the quaternary ammonium salt is present in a concentration of about 3 percent to about 8 percent by weight of the composition.

6. The composition of claim 1, wherein the combined concentration of the quaternary ammonium salt plus the starch is at least about 6.5 percent by weight, based on total weight of the composition.

7. The composition of claim 1, wherein the combined concentration of the quaternary ammonium salt plus the starch is about 6.5 percent to about 9 percent by weight, based on total weight of the composition.

8. The composition of claim 1 comprising less than about 7 percent by weight of electrolyte.

9. The composition of claim 1 having a pH of about 4.5 to about 8.

10. The composition of claim 1 having a total silicone concentration of less than about 3 percent by weight.

11. The composition of claim 1, wherein at least two of R₁, R₂, R₃, and R₄ of the quaternary ammonium salt comprise a hydrophobic moiety that includes an alkyl group and an ester functional group.

12. The composition of claim 1, wherein the starch is a hydroxypropyl starch phosphate.

13. The composition of claim 1 comprising less than about 15 percent by weight of hydrophobic agents.

14. A method of treating mammalian skin comprising applying the composition of claim 1 thereto.

15. The method of claim 14, further comprising allowing said composition to be left on said skin for at least an hour without rinsing.
